(19) United States
(12) Patent Application Publication
(10) Pub. No.: US 2004/0109866 A1
(43) Pub. Date: Jun. 10, 2004

(54) Method and composition

(76) Inventors: Nicola Jane Chumas, Harrogate (GB); Paul Dominic Chumas, Harrogate (GB)

Correspondence Address: Charles D Gunter Jr, Whitaker Chalk Swindle & Sawyer, 3500 City Center Tower II, 301 Commerce Street, Ft Worth, TX, 76102-4186, US

(21) Appl. No.: 10/471,816

(22) PCT Filed: Mar. 21, 2002
(86) PCT No.: PCT/GB02/01132

(30) Foreign Application Priority Data
Mar. 21, 2001 (GB) 0107033.3

(51) Int. Cl. A61K 39/395

(52) U.S. Cl. 424/178.1

(57) ABSTRACT

The present invention relates to an immunoactive composition of matter having the general formula AB(T) where AB is an antigen, growth factor or other material which binds to receptor sites on epithelial target cells, notably choroid plexus cells, and T is a moiety which affects the activity of the target cell, for example a toxin or medicament, and which is attached, notably by one or chemical linkages, to the moiety AB. The immunoactive material selectively attaches to the epithelial cells and the moiety T then is absorbed by those cells, thus reducing the risk of acting on other cells. The invention also provides a method of treating epithelial cells, notably the choroid plexus cells so as to regulate the production of CSF from those cells in the treatment of hydrocephalus, by administering the immunoactive material by injection or infusion into the CSF circulation system so as to localise the administration and action of the immunoactive material. The immunoactive material also finds use in the treatment of other epithelial cells, for example in the treatment of pituitary gland, renal or occular cells.

The present invention relates to a method and composition, notably to a method for treating conditions in the brain of a mammal, and to a composition for use in that method.

### BACKGROUND TO THE INVENTION

The brain of a human being or other mammal is bathed in cerebrospinal fluid (CSF). This CSF is largely produced by the choroid plexus, which is situated in the ventricles (lateral, third and fourth ventricles) of the brain. Conventional theories propose that the CSF circulates through the ventricular system before passing out of the fourth ventricle and circulating down the spinal canal and re-entering the head. The CSF then passes over the surface of the brain in the subarachnoid space, where it is absorbed into the venous system.

Studies have shown that an adult human produces approximately 20 mls of CSF per hour. Under healthy conditions, there is a balance between production and absorption of the CSF (the CSF dynamics), so that the intra-cranial pressure is kept within normal limits.

The brain does not have a lymphatic system and most of the extra-choroidal production of CSF is from extra-cellular fluid draining into the CSF pathways. In humans it is estimated that this extra-choroidal production makes up 10-30% of the CSF circulating around the brain.

There are many ways in which the CSF dynamics may become altered. The most common of these is termed hydrocephalus (“water on the brain”). In this condition there is a build-up of CSF resulting in dilation of the ventricular system and consequently a build up of pressure inside the head. CSF shunting devices, that is subcutaneous tubes which serve to drain CSF from the ventricular system to a distal body compartment, usually the abdomen, are currently used to drain away excess CSF and thus relieve the pressure within the head. Prior to the advent of such devices, hydrocephalus was often fatal and survivors were usually left severely disabled.

Although the use of shunts has revolutionised the treatment of hydrocephalus, it has been estimated that 30-40% of new shunts will require revision within the first year of insertion and 5-10% will need removal due to infection. In the USA about 125,000 shunt procedures are carried out annually at a cost of $100 million. There is thus a need for a non-surgical alternative to this expensive and often unsuccessful method for treating hydrocephalus.

We have now devised a method and composition which offer a non-surgical alternative to the use of shunts.

The choroid plexus cells are known to be epithelial in nature and thus to differ from other cells in the brain parenchyma, apart from the pituitary gland which also exhibits epithelial cells. Such cells express characteristic antigens or receptor sites, for example growth factor or, in the case of the pituitary gland hormone, receptor sites, on the surface of the cell. For convenience, such antigens and receptor sites are hereinafter generally denoted by the term epitopes. These epitopes differ from those expressed by other cells in the brain. However, these facts have not been used in the treatment of hydrocephalus. In the present invention, an antibody or growth factor which selectively binds to these epitopes is used to achieve binding of a toxin or other component attached to the antibody or growth factor, which other component is then absorbed by the target cell and alters the functioning of that cell. The component thus selectively acts upon the target choroid plexus cells and we have confirmed by staining tests that, using the antibody to epithelial membrane antigen (EMA) as the antibody to bind to the epithelial cell, there is little or no staining elsewhere in the brain parenchyma, thus confirming the selectivity of the binding of the antibody. This reduces the risk of side reactions between the other components and other cells within the brain. Furthermore, the other component can be administered directly into the CSF circulation system, thus localising the toxin or other component in the region of the choroid plexus cells. The activity of the toxin or other component is thus localised and its effect is not dissipated throughout the body. Furthermore, since the immunotoxin is administered into the CSF, it does not readily find its way to the liver and thus remains in the desired location without being broken down and excreted as with immunotoxins administered intravenously.

It has been proposed, see for example the review by Vitetta and Thorpe in Cell Biology Vol2 1991, pp47-58, to attach toxins to antibodies or growth factors to form immunotoxins for the treatment of cancer tumour cells by attachment of the antibody or growth factor to the epitopes expressed by the cancerous cells. However, problems are encountered with such treatment in that the antibody or growth factor carrying the toxin often binds to cells other than the desired target cells, and in that the immunotoxin is administered intravenously and is swept by the blood stream to the liver where it is broken down and the toxin excreted before it has been able to act upon the cancerous cells. Furthermore, the toxin can act upon a wide range of cells during its transport to the liver, bringing the risk of undesirable side effects. A further problem arises in that repeated administration of such immunotoxins may invoke an immune response from the patient.

In the present invention, the specificity of the antibody or the growth factor to the epitopes expressed by the epithelial choroid plexus cells, as opposed to other cells in the brain, is used to provide a selective attachment of components carried by the antibody or growth factor for the modification of the action of the target cells.

The epithelial choroid plexus cells express characteristic epitopes, notably EMA, Na/K ATP'ase and MDR, on the surface of the cells and these epitopes are not expressed on other brain tissue structures. In the case of the pituitary gland the characteristic epitope is hormonal.

The epitopes thus provide a unique and specific external identification of the choroid plexus cells and the epithelial cells of the pituitary gland and differentiate such cells from other brain tissue. Characterisation of these epitopes on the surface of the cells identifies the types of antibodies, interleukins or growth factors which will preferentially bind to those cells via those epitopes. We have found that some antibodies, notably MDR, binding to such epitopes can have a direct effect on the activity of the choroid plexus cells. Furthermore, if a toxin is attached to the antibody, interleukin or growth factor to form an immunotoxin, this then has the ability to selectively kill or disable the choroid plexus cells and hence decrease the production of CSF. Such a method for treating hydrocephalus by administration, for example by the intra-thecal (that is via the ventricles or the lumbar CSF), intra-vascular or oral routes, of an immunotoxin which is selectively and specifically taken up at the surface of the choroid plexus cells avoids the need for surgical methods of treatment, apart from the surgery necessary for the administration of the immunotoxin.

We believe that this approach may also be used to attach a medicament or other material to the epithelial choroid plexus cells to stimulate the generation of CSF, for example in Alzheimer's disease when CSF production is reduced. The invention thus provides a method for managing the production of CSF over a wide range of conditions. We further believe that the invention may also be applied to other epithelial cells elsewhere in the body.

### SUMMARY OF THE INVENTION

Accordingly, the present invention provides a method for the treatment of a condition in a mammal, notably for regulating the production of CSF in that mammal, involving the action of a target epithelial cell which expresses characteristic epitopes, characterised in that:

a. the method comprises administering to the target cell a pharmacologically effective amount of an immunoactive material; and in that

b. the immunoactive material contains an ingredient which affects the activity of the target cell; and in that that ingredient is, or is attached to, an antibody, interleukin or growth factor which will attach to an epitope expressed by the target cell,

whereby the said immunoactive material is selectively attached to the target cell and the said ingredient is absorbed by the target cell.

Preferably, the target cell is an epithelial choroid plexus cell and the ingredient attached to the antibody, interleukin or growth factor is a toxin to the target cell, a radionucleotide or a medicament.

The term immunoactive is used herein to denote a material which contains one or more antibodies, interleukins, growth factors, or the active fragments thereof, which will attach to the epitope expressed by the target epithelial cell, and which is or has attached to it an ingredient which affects the activity of the cell. In this context, therefore, the term immunoactive includes a material, for example a carbonic anhydrase inhibitor, which has a modulating effect on the activity of the epithelial cells; as well as one which acts to disable or kill the target cell. Whilst some forms of antibody, notably MDR, may themselves have the desired effect upon the activity of the target cell, it is preferred that the antibody or growth factor carry attached thereto a component which provides or enhances the desired effect upon the target cell. It is preferred that such a component be a toxin, such as A chain ricin and/or whole ricin (IT-R), which disables or kills the cell. For convenience the invention will be described hereinafter in terms of the use of an immunotoxin which comprises an antibody having attached thereto such a toxin.

Thus, from another aspect, the present invention provides a method for the treatment of a condition in a mammal involving an epithelial cell which expresses one or more epitopes which are uniquely characteristic of that cell, which method comprises administering to that mammal a pharmacologically effective amount of an immunotoxin comprising an antibody, growth factor or a fragment thereof, to which is attached a toxin to that epithelial cell, which immunotoxin attaches selectively to that epitope expressed on the surface of that epithelial cell.

As indicated above, the method of the invention can be applied to the treatment of a wide range of conditions involving the action of an epithelial cell: for example the treatment of ophthalmic conditions, notably glaucoma by disabling the sodium/potassium pump action of the fluid producing cells in the eye; the reduction of CFS production in the case of injuries to the head or benign intracranial hypertension; the treatment of primary choroid plexus tumours and other tumours such as dermoid tumours, epidermoid tumours; the treatment of meningiomas, teratomas, germ cell tumours, craniopharyngiomas, and metastatic carcinomas to the central nervous system which all may express epithelial antigens. Pituitary tumours have epithelial cells and frequently have hormone receptors which act as the epitopes for use in the method of the invention. Ependymomas expressing the MDR antigen may also be treated using the method of the invention. The invention may also be applied to renal tissues, for example the treatment of renal tumours. However, the invention is of especial application in regulating the production of CSF by increasing or decreasing the production of CSF by the activity of the epithelial choroid plexus cells, since the preferred method of administration of the immunoactive material via the CSF circulation system is particularly efficacious in such application. For convenience, the invention will be described hereinafter in terms of this preferred application.

We believe that the immunoactive materials for use in the present invention are novel and the invention therefore also provides a composition of matter suitable for use as the immunoactive material in the method of the invention, which material has the general formula:

AB(T) wherein

AB is an antibody, interleukin or growth factor moiety, or a fragment thereof, which is capable of binding to a epitope expressed on the surface of an epithelial choroid plexus cell; and

T is a moiety which can be absorbed by the said cell and which affects the activity of the cell; and

( ) denotes an attachment between the moieties AB and T.

As indicated above, the choroid plexus cells express characteristic epitopes on their surface which are specific to epithelial cells. These epitopes include antigen and growth factor receptor sites such as IgG, GFAP, HMFG1, TTR, ZO-1, cytokeratin, cadherin family, catenin family, vimentin family, SV40, EMA, GA733-2E, galectin family, Ep-CAM, EGP40, mucin family, CD44, claudin family, lectin family, Fas, carbohydrate Ag family (CA19-9, Le(Y)), Span-1, KMO-1, Human milk fat globulin, secretory component, BerEP4, HEA125, epithelial glycoprotein-2 (EGP-2), Thy1.1 antigen, and neural cell adhesion molecule; and growth factor receptor sites such as fibroblastic growth factor, epidermal growth factor receptor, neurotrophic growth factor/receptor and erbB2-receptor. The epitope may also be an interleukin receptor.

In diseased states, some atypical epitopes may be expressed or the relative proportions of typical epitopes expressed on the epithelial cell surface may be different from that normally encountered. In such a case it may be desired to characterise such atypical expressions to identify the nature of the reactive sites on the peptide chains thereof to identify the types of antibody which would bind to them in the method of the invention. The characterisation of the epitopes can be carried out using any suitable technique. For example, a sample of the choroid plexus cells may be obtained from the patient by a conventional biopsy or by sampling the CFS in the ventricular system and assaying the nature of the reactive sites thereon using a suitable technique, for example by passing the sample of the cell culture or an extract thereof through a chromatographic column.

For convenience, the invention will be described hereinafter in terms of an epithelial choroid plexus cell having a single expressed epitope with which a single immunoactive material attaches. It is particularly preferred to use the epitopes IgG, EMA, Na/K ATP'ase and/or MDR as the receptor sites to which to attach the immunoactive materials.

In the present invention, an antibody and/or growth factor and/or interleukin moiety AB is used to attach the moiety T to the epitope of the epithelial cell. The AB moiety is selected from any suitable antibody, interleukin or growth factor and typically includes antibodies to EMA, HMGG1, E-cadherin, β catenin or EGF. A particularly preferred moiety AB for present use is the antibody to the EMA antigen.

If desired a fragment of an antibody or growth factor may be used as the moiety AB, as it has been found that such fragments often enhance the efficacy of the immunotoxin due to their reduced size. Typical of such fragments for present use include Fab′ or F(ab′).

If desired, mixtures of the moieties AB may be used and pharmacologically acceptable derivatives or precursors of such moieties may be used, for example to form the desired moiety AB in situ during the formation of the immunoactive compound.

Many suitable antibodies, interleukins or growth factors, both natural and synthetic, suitable for use as the moiety AB are commercially available and may be used in the present invention in their commercially available forms.

The moiety T of the immunoactive material can be selected from a wide range of materials which are absorbed by the cell, for example by coupling of part of the T moiety of the immunoactive material with glycoproteins or glycolipids expressed on the surface of the target cell and absorption of the moiety T or an active component thereof through the cell wall. As stated above, the moiety T may be one which disables or kills the cell, for example a virus, whole ricin or A chain ricin; or could be a medicament. which increases the CSF production of the cell, or interferes with ion channels in the cell, for example the NA/K pump and associated channels, the Chloride channels, or the various Calcium channels. Such drugs include (but are not limited to) acetazolamide, verapamil, calcium agonists and antagonists. The moiety T may be a material which affects the activity of the cell by some other means, for example by irradiation of the cell as when a radionucleotide is used as the moiety T. However, it is particularly preferred to use a material which kills or disables the choroid plexus cell, for example the toxins ricin, botulinum, pseudomonas exotoxin, anti-CD22-A or saporin; or a cancer chemotheraputic agent, or Cyclacel. Such toxins may be natural or synthetic and may be genetically modified or otherwise treated to enhance their activity.

Typically, the AB and T moieties will be peptide molecules or residues thereof and the chain of the peptide will present sites at which chemical or other linking can take place. The moieties AB and T can be attached to one another by any suitable technique. Thus, in the case of ricin, attachment will usually be via a disulphide bond connecting the A and B chains of the whole ricin, which may have been activated by, for example, removal of the B chain of whole ricin. Such techniques are known in the art and include, for example derivatisation of the moiety AB using SMTP and reduction of the moiety T using DDT followed by their reaction to form an inter-chain disulphide bond linking them, the introduction of a thiol group into the moiety AB where not already present and an alkylating function into the toxin moiety T, which then react to form a thioether linkage. Alternatively, a free cysteine residue in the moiety AB, notably the fragment forms Fab/Fab′, may be used to form the thioether linkage with a disulphide linkage in the moiety T using Ellman's reagent.

It will be appreciated that where the epitope and/or the moiety AB and/or the moiety T do not possess reactive sites by which they can be directly attached to one another, it may be necessary to use a further material which acts as a bridging component to link the incompatible materials. Such bridging components, for example heterobifunctional groups such as SSPy, SPDP and SMTP, and their mode of operation are known in the art and may be used in the conventional manner.

Thus, the term “attached” as denoted by the symbol ( ) in the general formula given above for the immunoactive material is used herein to denote that the moiety T is carried by the moiety AB and that the moiety AB is carried by the epitope, either by being chemically bound thereto through mutually reactive sites therein or via an intermediate bridging moiety L which provides suitable reactive sites to bind the moieties where suitable reactive sites are not inherently present in the moieties AB, T and/or the epitope.

The optimum technique and conditions for forming the immunoactive will depend upon the nature of the individual moieties and the nature of the attachment which is to be formed between them and can readily be determined by simple trial and error tests following identification of the optimum forms of the moieties for present use.

The immunoactive material will typically contain the. moieties AB and T in molar ratios of from 05:1 to 2:1, preferably about 1:1, and a molar excess, typically from 10 to 100% excess, of the moiety T will usually be used in the production of the immunoactive material. Excess of the moiety T may be removed from the immunoactive material product by any suitable technique.

The resultant immunoactive material may be used in the form in which it is obtained without isolation from the medium in which it is produced. However, it may be desired to isolate and purify the immunoactive material using any suitable technique, for example using affinity chromatography on Blue Sepharose to remove free moieties AB and gel filtration of Sephacryl S-200HR to remove high molecular weight material and freetoxin.

For convenience, the invention will be described hereinafter in terms of an immunoactive material which has been isolated from the medium in which it has been produced and which has subsequently been put up in an aqueous or other medium appropriate to the method to be used for the administration of the immunoactive material to a patient. If desired, the isolated immunoactive material may be dried, for example freeze dried or dried in the presence of trehalose, to form a dry product suitable for storage and subsequent reconstitution as an aqueous solution or suspension of the desired concentration for administration to a patient.

If desired, the composition containing the immunoactive material may contain other ingredients to enhance the performance of the immunoactive material. Thus, for example, the immunoactive material may be used in combination with a lysosomotropic amine such as ammonium chloride or chloroquine, or a carboxlic ionophore.

The composition containing the immunoactive material can be administered to the patient by injecting a suitable quantity of an aqueous composition containing the immunoactive material into the circulating CSF fluid, for example by injection into the cerebral ventricles or the spinal CSF. Alternatively, the immunoactive material may be applied by oral, intravenous or intra-arterial administration. However, it is preferred to administer the immunoactive material by direct injection into the CFS circulation system so as to localise the administration of the immunoactive material in the neighbourhood of the cells to be treated.

The optimal route and dosage can readily be determined for each case and we have found that effect of the immunoactive material is usually directly related to the dosage administered. This can be progressively increased to the level at which a satisfactory response from the patient is achieved or to just below that at which the patient exhibits adverse side reactions to the immunotoxin. It is within the scope of the invention to apply the composition containing the immunotoxin by infusion to achieve prolonged administration. This could be achieved by regular injections into the CSF directly, via an implanted access device, or via an external or implanted pump system, in order to administer the immunotoxin over a period of time. The best method for applying the immunotoxin will depend upon the specific circumstances of each case and may be determined by simple tests using conventional techniques.

The invention has been described above in terms of the treatment of hydrocephalus or other conditions which result in an imbalance in CSF production/absorption/dynamics. However, it will be appreciated that the invention can be applied to other conditions. Such other conditions include all forms of hydrocephalus, benign intracranial hypertension and other causes of raised intracranial pressure, for example head injuries. The invention can also be applied to the killing of malignant cells, for example primary choroid plexus tumours and other intra-venticular tumours, for example ependymomas; and meningiomas; and in the treatment of tumours of the pituitary gland. Renal tissue and parts of the eye have areas which have similar cells to those seen in the choroid plexus and the invention may be of use in renal diseases, for example renal tumours, and in ophthalmic conditions, for example glaucoma.

The invention will now be described by way of illustration with respect to preferred embodiments of the invention as shown in the following examples in which all parts and percentages are given by weight unless stated otherwise. In the accompanying Figures,

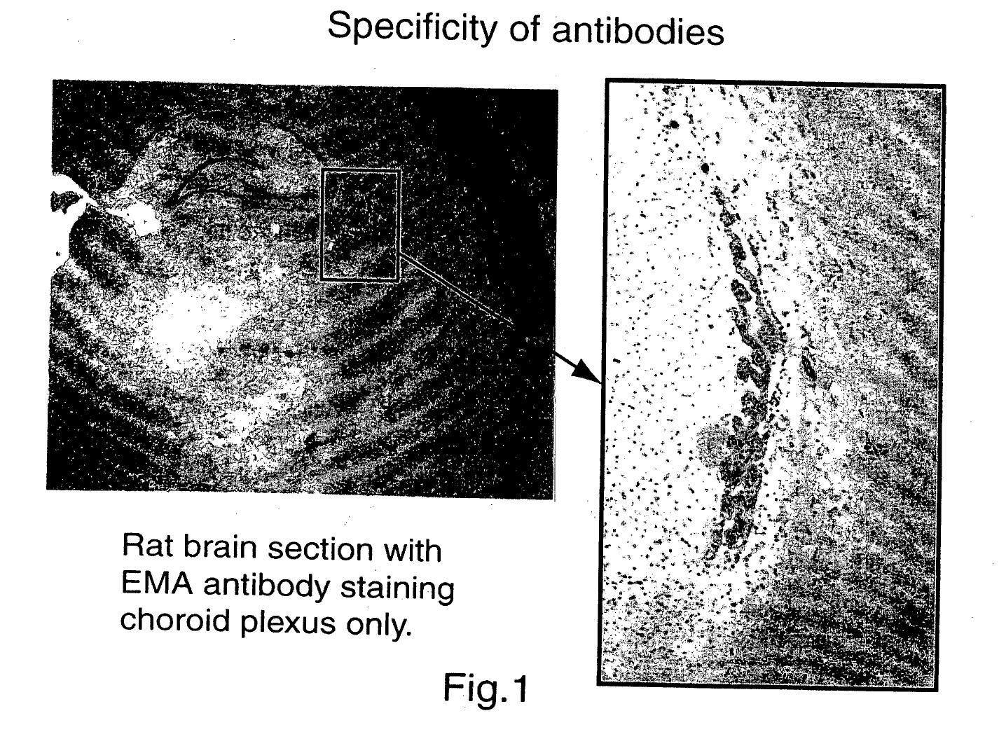

FIG. 1 shows the selectivity of attachment of the antibody EMA to the epithelial choroid plexus cells in a rat brain;

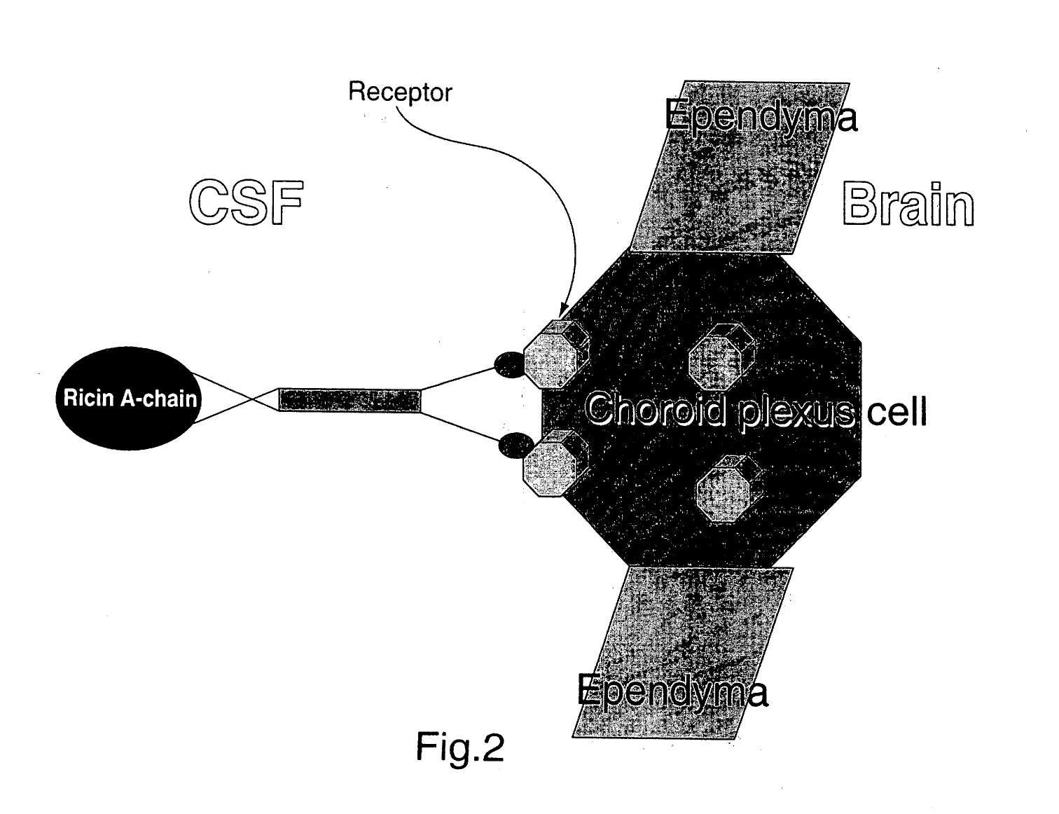

FIG. 2 is a diagrammatic representation of an immunoactive material of the invention attaching to receptor sites on a choroid plexus cell;

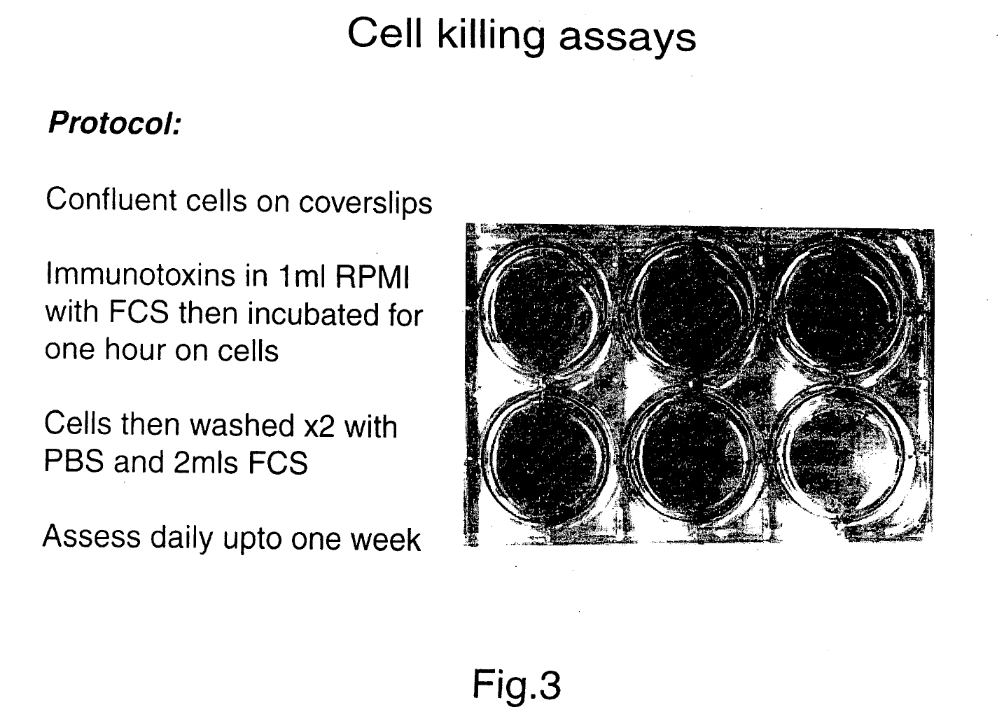

FIG. 3 shows the protocol for assessing the effect of the immunoactive material on cells;

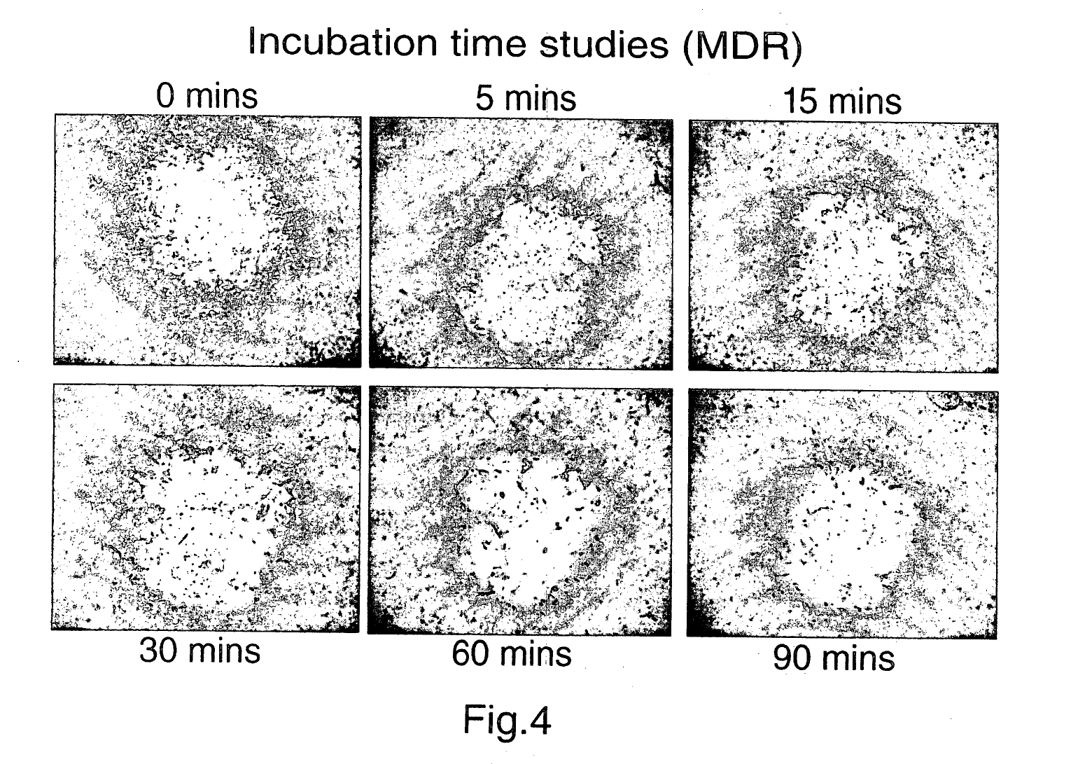

FIG. 4 shows a series of photographs of one of the assays from FIG. 3 using MDR as the immunoactive material taken at the stated intervals from application of the immunoactive material to the choroid plexus cells, clearly demonstrating that the material is effective rapidly in killing the cells; and

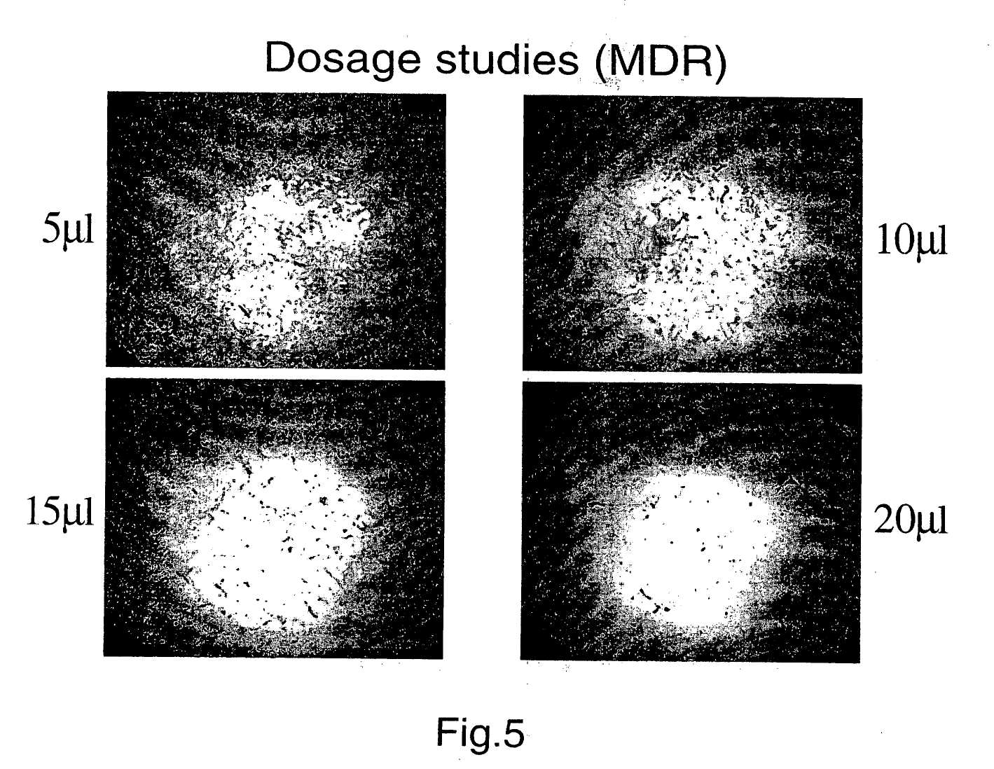

FIG. 5 shows a series of photographs of one of the assays from FIG. 3 using different dosages rates of the immunoactive material, clearly demonstrating the dose dependent effect of the material.

### Three commercially available epitopic antibody peptide fragments were selected as antibodies to the following antigens:

1. Na-K ATP'ase Beta 1 subunit—found in the ectodomain of the Beta subunit (approx aa 62-304)—available from Research Diagnostic Inc., Flanders, N.J., U.S.A. This antigen was chosen because it is known that choroid plexus cells are rich in Na-K ATP'ase. It may have a role in the treatment of glaucoma.

2. The multi-drug transport protein known as P-glycoprotein, 0170 or MDR1. This plasma membrane bound protein has both cytoplasmic and extracellular domains and the antibody was chosen since it specifically recognises the extracellular component—obtained from ImmunoKontact, AMS Biotechnology, Abingdon, Oxfordshire, England.

3. EMA (Epithelial Membrane Antigen). This is a well recognised epithelial antigen and our preliminary studies in rat brains showed specific uptake in the choroid plexus with no uptake in the brain parenchyma—see FIG. 1.

However, as stated above, other suitable antigen/peptide fragments could be obtained by further characterising the antigens expressed on the choroid plexus cell surface and synthesising antibodies for those antigens.

The toxin used was Ricin A chain which was bound to the peptide chain of the antibody using the ligands S-MBS and SDPD—manufactured by Pierce Pharmaceuticals.

Immunotoxin Manufacture

The plant-derived agent ricin, a potent inhibitor of protein synthesis, was used as the toxin used for the present example. However, many other agents which either kill the cell or interfere with cell function could be used. Ricin is a glycosylated holotoxin consisting of targeting (B-chain) and toxin (A-chain) sub-units. In the absence of B-chain, the toxicity of ricin A-chain is eliminated both by removal of the sugar moieties and because it is unable to enter the target cell. Ricin-based immunotoxins generally consist of ricin A-chain linked to a specific antibody, with the antibody fulfilling the targeting role of the ricin B-chain. Antibody (Na/K ATP'ase, MDR1, EMA or IgG) is conjugated to the deglycosylated A-chain of ricin (RCA), and purified from unconjugated toxin using standard protocols. Thus, RCA (obtained from Sigma Chemical Co) is incubated with the bifunctional cross-linking reagent m-maleimidobenzoyl-N-hydroxysuccinimidyl (MBS) ester (Pierce Chemical Co) before mixing with a fresh preparation of partially reduced monoclonal antibody. The activated RCA reacts with and becomes bound to the reduced antibody, creating a specific immunotoxin (ITX). To remove unconjugated toxin, the reaction product is subjected to FPLC using a size-exclusion column (Pharmacia) and fractions containing only ITX (as assessed by apparent molecular weight) are collected. If desired, unconjugated antibody can be removed by binding the ITX to an immobilised D-galactose affinity column (Pierce), flushing the column with PBS then eluting the ITX with a lactose solution.

In vitro cell culture work was undertaken using a sheep choroid plexus cell line and a human choroid plexus carcinoma cell line. Initial work confirmed the selective presence of EMA, Na-K ATP'ase and MDR1 on the surface of the choroid plexus cells. In the experiments the immunotoxin produced as described above was placed on the cells for one hour and then was washed off with calf-serum. Cell death was watched over the next 24 hours to 1 week, depending on the immunotoxin and dose of immunotoxin used. Cell death varied from partial, as seen with the IgG immunotoxin, through to virtually complete, as seen with the EMA, Na/K ATP'ase and MDR1 immunotoxins. If desired, an adjunct, for example chloroquine which raises lysosomal pH and increases the efficacy of the toxin in the immunotoxin may be used in admixture with the immunotoxin applied to the choroid plexus cells.

The reduction in live cells in these tests demonstrated the efficacy of the immunotoxin in selectively killing or disabling the choroid plexus cells, as shown in FIGS. 4 and 5, and thus their capacity to reduce the production of CSF. This was compared to a control test in which antibody was administered without being attached to the RCA on sheep choroid plexus cells and in which little or no cell death occurred.

Further studies have shown that this cell-death is in a dose-dependant fashion and is specific for choroid plexus cells, as shown in FIG. 5. This result demonstrates that it is possible to “titrate” the amount of immunotoxin delivered in vivo to a level where CSF production is reduced sufficiently to control symptoms without killing all the choroid plexus.

In an alternative test, MDR1 antibody was used without being attached to the RCA. The MDR1 antibody alone effected significant cell death when applied to choroid plexus cells. The results of this test are shown in FIGS. 4 and 5.

1. A composition of matter suitable for use as the active ingredient of an immunoactive material, which composition has the general formula:
AB(T) wherein
AB is an antibody, interleukin or growth factor moiety, or a fragment thereof, which is capable of binding to a epitope expressed on the surface of an epithelial choroid plexus cell; and
T is a moiety which can be absorbed by the said cell and which affects the activity of the cell; and
( ) denotes an attachment between the moieties AB and T.

2. A composition of matter as claimed in claim 1, characterised in that the moiety AB is the antibody or growth factor for one or more of the epitopes IgG, GFAP, HMFG1, TTR, ZO-1, cytokeratin, cadherin family, catenin family, vimentin family, SV40, EMA, GA733-2E, galectin family, Ep-CAM, EGP40, mucin family, CD44, claudin family, lectin family, Fas, carbohydrate Ag family (CA19-9, Le(Y)), Na/K ATP'ase, Span-1, KMO-1, Human milk fat globulin, secretory component, BerEP4, HEA125, epithelial glycoprotein-2 (EGP-2), EGF, Thy1.1, neural cell adhesion molecule, fibroblastic growth factor, epidermal growth factor, neurotrophic growth factor, erbB2, interleukin or MDR.

3. A composition of matter as claimed in claim 1, characterised in that the antibody is the antibody for the epitopes EMA, Na/K ATP'ase, HMGG1, E-cadherin, β-catenin, EGF and/or MDR.

4. A composition of matter as claimed in either of claims 1 or 3, characterised in that the antibody moiety AB is used as a fragmented form thereof.

5. A composition of matter as claimed in claim 4,. characterised in that the fragmented form of the moiety AB is Fab and/or Fab′.

6. A composition of matter as claimed in any one of the preceding claims, characterised in that the moiety T is selected from a toxin for epithelial cells, a radionucleotide, or a drug which modifies the activity of the epithelial cell.

7. A composition of matter as claimed in claim 6, characterised in that the moiety T is a virus, acetazolamide, verapamil, a calcium antagonist or agonist, cyclacel, whole ricin, A chain ricin, botulinium, pseudomonas exotoxin, anti-CD22-A, saporin, or mixtures or precursors or derivatives thereof.

8. A composition of matter as claimed in any one of the preceding claims, characterised in that the represents a chemical linkage between the moieties AB and T.

9. A composition of matter as claimed in claim 8, characterised in that the linkage is via a thioether linkage.

10. A composition of matter as claimed in any one of the preceding claims, characterised in that the attachment ( ) is via an intermediate bridging component.

11. A composition of matter as claimed in claim 10, characterised in that the intermediate component is SSPy, SPDP or SMTP.

12. A composition of matter as claimed in any one of the preceding claims wherein the moieties AB and T are present in molar proportions of from 0.5:1 to 2:1.

13. A composition of matter as claimed in claim 1, substantially as hereinbefore described in the examples.

14. A composition of matter as claimed in claim 1, characterised in that it is put up as an aqueous composition suitable for administration to a mammal by injection or infusion.

15. A method for preparing a composition as claimed in any one of the preceding claims, characterised in that a moiety AB is reacted with a moiety T to form a sulphide or thioether linkage between the two moieties.

16. A method as claimed in claim 15, characterised in that the moiety AB is derivatised using SMTP and the moiety T in reduced using DDT and the products reacted to form a disulphide bond linking the moieties AB and T.

17. A method as claimed in claim 15, characterised in that a thiol group is introduced into the moiety AB, where this is not already present, and the moiety T is alkylated and the products reacted to form a thioether linkage between the moieties AB and T.

18. A method as claimed in claim 15, substantially as hereinbefore described in the examples.

19. A method for the treatment of a condition in a mammal involving the action of a target epithelial cell which expresses characteristic epitopes, characterised in that:
c. the method comprises administering to the target cell a pharmacologically effective amount of an immunoactive material; and in that
d. the immunoactive material contains an ingredient which affects the activity of the target cell; and in that that ingredient is, or is attached to, an antibody, interleukin or growth factor which will attach to an epitope expressed by the target cell,
whereby the said immunoactive material is selectively attached to the target cell and the said ingredient is absorbed by the target cell.

20. A method of treatment as claimed in claim 19, characterised in that the target cell is an epithelial choroid plexus cell and the immunoactive material acts to modify the production of CSF from that cell.

21. A method of treatment as claimed in either of claims 19 or 20, characterised in that the immunoactive material incorporates a toxin for the target cell, a radionucleotide and/or a medicament or mixtures thereof.

22. A method for the treatment of a condition in a mammal involving an epithelial cell which expresses one or more epitopes which are uniquely characteristic of that cell, which method comprises administering to that mammal a pharmacologically effective amount of an immunotoxin comprising an antibody, growth factor or a fragment thereof, to which is attached a toxin to that epithelial cell, which immunotoxin attaches selectively to that epitope expressed on the surface of that epithelial cell.

23. A method as claimed in either of claims 19 or 22, characterised in that the immunoactive material is a composition of matter as claimed in any one of claims 1 to 14.

24. A method of treatment as claimed in any one of claims 19 to 23, characterised in that treatment is for regulating the production of CSF from the epithelial choroid plexus cells.

25. A method of treatment as claimed in any one of claims 19 to 23, characterised in that the immunoactive material is administered as an aqueous composition by injection or infusion into the circulation system of the CSF of a human patient.

26. A method of treatment as claimed in any one of claims 19, characterised in that the target cell is a pituitary gland cell.
